(19) United States
(12) Patent Application Publication
(10) Pub. No.: US 2025/0084479 A1
(43) Pub. Date: Mar. 13, 2025

(54) METHODS AND COMPOSITIONS FOR DETERMINING THE ANTIGEN SPECIFICITY OF T CELLS

(71) Applicants: The Broad Institute, Inc., Cambridge, MA (US); The General Hospital Corporation, Boston, MA (US)

(72) Inventors: Nir Hacohen, Cambridge, MA (US); Matthew Bakalar, Cambridge, MA (US)

(73) Assignees: The Broad Institute, Inc., Boston, MA (US); The General Hospital Corporation, Boston, MA (US)

(21) Appl. No.: 18/961,295

(22) Filed: Nov. 26, 2024

Related U.S. Application Data
(63) Continuation of application No. PCT/US2023/067706, filed on May 31, 2023.
(60) Provisional application No. 63/347,517, filed on May 31, 2022.

(51) Int. Cl. C12Q 1/6881; C07K 1/22; C07K 14/195; C07K 14/74; C12N 1/20; C12N 7/00; C12N 9/02; C12N 9/64; C12N 9/90; C12P 21/00

(52) U.S. Cl. CPC C12Q 1/6881; C07K 1/22; C07K 14/195; C07K 14/70539; C12N 1/20; C12N 7/00; C12N 9/0051; C12N 9/6472; C12N 9/90; C12P 21/00; C07K 2319/95; C12N 2795/18123; C12N 2795/18151; C12Y 108/03002; C12Y 304/22068; C12Y 503/04001

(57) ABSTRACT

The present disclosure provides methods and compositions for determining the antigen specificity of T cells and in a scalable, high-throughput approach. The disclosure provides methods for producing RNA-barcoded pMHC multimers that can be decoded using single-cell RNA sequencing methods. Among these, disclosed herein are multivalent virus-like-particles bound with pMHC in E. coli cells that encapsulate an RNA barcode encoding the peptide identity.

### RELATED APPLICATIONS

This application claims the priority under 35 U.S.C. § 119(e) to U.S. Provisional Application U.S. Ser. No. 63/347,517, filed May 31, 2022, which is incorporated herein by reference.

### REFERENCE TO AN ELECTRONIC SEQUENCE LISTING

The contents of the electronic sequence listing (B119570167WO00-SEQ-JQM.xml; Size: 14,114 bytes; and Date of Creation: May 31, 2023) is herein incorporated by reference in its entirety.

### BACKGROUND OF THE INVENTION

T cells play a central role in cancer immunotherapy but determining their antigen-specificity remains extremely challenging experimentally and is not yet feasible using computational approaches (Peters, Nielsen, and Sette 2020). Next-generation sequencing has made it possible to directly determine the primary sequence of the T cell receptor (TCR), but the structural diversity of the TCR and the polymorphism of major histocompatibility complex (MHC) genes have precluded direct prediction of the peptide-MHC-TCR complex (designated as “pMHC-TCR”).

Moreover, the number of directly validated pMHC-TCR interactions (ranging in the thousands) is miniscule compared to the size of the human TCR repertoire (ranging in the high millions based on recent measurements of the beta chain alone, but multiplicatively more based on random combinations of both chains (Qi et al. 2014)). Thus, there is a critical need for scalable methods for discovering pMHC-TCR interactions that can then be used to train generalized, predictive models of pMHC-TCR binding using machine learning. These predictive models can then be used to determine if TCRs found in patients are reactive to tumor antigens predicted from sequence data.

Existing approaches are inadequate for experimentally discovering matched TCRs and cognate peptides, and are limited to (1) searching for peptides that match a single TCR (one TCR to many peptides), or (2) searching for TCRs that bind one peptide (one peptide to many TCRs). To date, only 105 peptides have been mapped to at least 50 TCRs per peptide (Chronister et al. 2021), which is hypothesized to be the minimum number of TCRs needed to classify TCRs that bind a particular pMHC. While various techniques have been used to detect antigen-specific T cells (e.g., yeast surface-display of a pMHC library to identify peptides that bind purified TCR-tetramers), these techniques are limited and cannot be easily scaled. At this time, there is no flexible, high-throughput method for mapping a pool of T cells to a library of peptides (i.e., mapping many TCRs to many pMHCs) to discover pMHC-TCR interactions.

### SUMMARY OF THE INVENTION

The present disclosure provides methods and compositions for determining the antigen specificity of T cells and in a scalable, high-throughput approach. Discovery of antigens for a population of disease-associated T cells remains difficult despite advances in TCR profiling. Disclosed herein in one aspect are methods for producing natively folded peptide-loaded MHC class I complexes in E. coli. In various aspects, the method is compatible with standard plasmid-based protein library production techniques, which makes it possible to generate libraries of greater than 10⁹ unique pMHC complexes in a pooled format. By including a multivalent scaffold (e.g., virus-like particles (VLPs) or self-assembling protein nanocompartments (e.g., protein nanocages)) for pMHC assembly in the expression constructs, highly diverse libraries of uniform (same peptide sequence per multimer) pMHC multimers (i.e., tetramers, pentamers . . . ) that are capable of binding to T cells through multivalent interaction with a cognate TCR can be produced.

The disclosure further provides methods for producing RNA-barcoded pMHC multimers that can be decoded using single-cell RNA sequencing methods. Among these, disclosed herein are multivalent virus-like-particles (VLPs) bound with pMHC in E. coli that encapsulates an RNA barcode encoding the peptide identity. The disclosure demonstrates the ability of these VLPs to bind specifically to cognate T cells with the sensitivity of classic tetramers. Single-cell RNA sequencing of VLP-bound T cells can be used to simultaneously recover T-cell receptor V(D)J sequences and cognate antigen sequences. The methods are accessible to a standard laboratory with molecular biology expertise, and VLP yield can be scaled inexpensively, resulting in a flexible platform for DNA-programmable interrogation of T cell specificity at unprecedented throughput and scale.

Without limitation, the applications for this disclosure in the area of T cell antigen discovery are numerous and include (1) identification of tumor-specific TCRs and their cognate peptides, (2) identification of TCRs in infectious disease and autoimmunity, (3) deep-profiling of the specificity of a single or set of TCRs. Finally, the compositions and methods disclosed herein provide for the ability to collect large-scale datasets pairing TCRs and cognate peptides, constructing a dataset that can be used to develop predictive computational models of TCR-peptide specificity.

Various aspects of the disclosure include, but are not limited to, the following:

Methods for producing properly folded MHC complexes in the cytoplasm of E. coli.

Methods for producing peptide-loaded HLA class I in the E. coli cytoplasm in which a peptide of interest is co-expressed and enzymatically processed to epitope length for loading onto MHC.

Methods for producing libraries of pMHC using plasmid libraries encoding multiple epitope peptides.

Methods for producing pMHC multimers (i.e., tetramers, pentamers etc.) loaded with a peptide of interest, including libraries thereof, in the E. coli cytoplasm.

A method for profiling the peptide-specificity of an MHC allele of interest by generating libraries of pMHC multimers and subsequently eluting peptides from purified pMHC for MS/MS analysis.

Methods for producing RNA-barcoded pMHC multimers, including libraries thereof, in the E. coli cytoplasm.

Methods for producing virus-like-particles decorated with pMHC loaded with a peptide-of-interest and encapsulating an RNA barcode, including libraries thereof.

Methods for utilizing the protein and nucleic acid compositions described herein for labeling antigen specific T cells via interaction with the T cell receptor.

Single-cell RNA sequencing of single T cells bound with pMHC VLPs (or RNA-barcoded pMHC multimers) displaying cognate antigen in order to pair TCR sequences paired with cognate peptide identity.

Accordingly, the present disclosure solves the critical need for scalable methods for discovering pMHC-TCR interactions that can then be used to train generalized, predictive models of pMHC-TCR binding using machine learning. These predictive models can then be used to determine if TCRs found in patients are reactive to tumor antigens predicted from sequence data.

In various aspects, the present disclosure provides:

A method for producing complexes between an antigen peptide and a major histocompatibility complex, comprising:
(a) providing an Escherichia coli cell under conditions suitable for expression, wherein the E. coli cell comprises one or more sequences encoding a major histocompatibility complex (MHC), one or more sequences encoding one or more heterologous oxidation enzymes, and a sequence encoding an antigen peptide; and
(b) isolating complexes between the antigen peptide and MHC (pMHC) from the E. coli cell.

A viral-like particle (VLP) conjugated to pMHC, comprising:
(a) a VLP composed of a self-assembling coat protein;
(b) a major histocompatibility complex (MHC); and
(c) an antigen peptide complexed with the MHC (pMHC),
   wherein the self-assembling coat protein and MHC are modified by fusion to a binding protein and binding peptide, respectively, and wherein the binding protein conjugates to the binding peptide.

A method for producing viral-like particles (VLPs) conjugated to pMHC, comprising:
(a) providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide; and
(b) isolating VLPs conjugated to complexes between the antigen peptide and MHC (pMHC) from the E. coli cell

A method for identifying pMHC-TCR pairs, comprising:
(a) providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide;
(b) isolating VLPs conjugated to complexes between the antigen peptide and MHC (pMHC) from the E. coli cell;
(c) contacting the isolated VLPs with a population of T cells;
(d) sequencing the population of T cells; and
(e) determining pMHC-T cell receptor (TCR) cognate pairs by identifying TCR-encoding sequences and antigen peptide-encoding sequences comprised by each T cell within the population of T cells.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1: Schematic illustrating T cell cytotoxicity. Cancer cells synthesize cancer antigens which are bound by MHC to produce MHC/antigen complexes (MHC/Ag) that are secreted to the cell surface. Extracellularly facing MHC/Ag is bound by T cells through specific TCRs, causing T cells to effector release phase when they release cytotoxic granules. Reproduced from de Charette et al. “Turning tumour cells into antigen presenting cells: The next step to improve cancer immunotherapy?” Eur J Cancer. 2016; 68:134-147. Copyright© 2016 Elsevier Ltd. All rights reserved.

FIG. 2: Schematic illustrating a method for determining peptide-MHC-TCR complex (pMHC-TCR) pairs. In step A, a plasmid library is prepared that encodes a beta-2 microglobulin human leukocyte antigen (B2M-HLA) heterodimer (a human MHC class I complex), as well as a ubiquitin-like-specific protease 1 (Ulp1), a protein disulfide isomerase (PDI), and a mitochondrial FAD-linked sulfhydryl oxidase (Erv1). The plasmid library further encodes a peptide that is unique to each plasmid and is fused to a ubiquitin-like protein, SMT3. In step B, plasmids of the HLA-plasmid library are transfected into E. coli cells expressing a bacteriophage viral coat protein (e.g., MS2, PP7). Each E. coli cell uniquely expresses a single SMT-peptide fusion protein which is cleaved by Ulp1, generating a peptide that is loaded onto B2M-HLA, thereby producing peptide-MHC complexes (pMHC). pMHCs are covalently incorporated into virus-like particles (VLPs) formed by assembly of the viral coat protein, due to protein/peptide tags encoded at the C-termini of the MHC and viral coat protein (e.g., SpyTag and SpyCatcher; DogTag and DogCatcher). Each E. coli is further transfected with a known RNA barcode oligonucleotide, which is packaged within the VLPs. In step C, E. coli cells are lysed and VLPs are collected. Each VLP is associated with a distinct pMHC and contains a unique RNA barcode. In step D, VLPs are panned with a population of polyclonal T cells, resulting in binding of pMHC on VLPs to T cell receptors (TCRs) on the surface of the T cells. In step E, T cells are sorted and subjected to single cell sequencing. RNA barcode sequencing is used to identify which peptide is bound to MHC (B2M-HLA) on the surface of the TCR-bound VLP, thereby establishing pMHC-TCR pairs.

FIGS. 3A-3B: Method for producing and profiling pMHC libraries in E. coli. FIG. 3A: Selected peptides are encoded in a pooled library and cloned into the HLA-plasmid library; Peptide-loaded HLA is expressed and assembled in the E. coli cytoplasm; Purification of pMHC library using affinity chromatography, followed by peptide elution; LC-MS/MS profiling of eluted peptides. FIG. 3B: DNA-programmed production with tandem mass spectrometry (MS/MS) results from 1,000 peptide HLA-A0201 library to demonstrate parallel profiling of peptide-HLA binding specificity.

FIGS. 4A-4C: Thermal stability of HLA-bound peptides. FIG. 4A: Schematic illustrating method for producing and profiling pMHC peptide thermostability. Selected peptides are encoded in a pooled library and cloned into the HLA-library plasmid; Peptide-loaded HLA is expressed and assembled in the E. coli cytoplasm; Purification of pMHC library using affinity chromatography, followed by treatment of up to 10 samples across a heat gradient and collection of eluted peptides; eluted peptides are barcoded with tandem-mass-tags (TMT); LC-MS/MS profiling of TMT labeled samples. FIG. 4B: Selected thermostability curves from 50 HLA-A2 bound peptides. FIG. 4C: Histogram of melting temperature (Tm) fit of the logistic function to thermostability curves.

FIGS. 5A-5C: Assembly of SpyTag-labeled pMHC-VLPs for screening. FIG. 5A: Composition of pMHC-VLPs that are self-assembled from multiple protein constructs inside the cytoplasm of E. coli cells. FIG. 5B: Specific binding of H2Kb-SIINFEKL VLPs to OT-I mouse CD8+ T cells. Flow analysis shows detection of cognate OT-I T cells but not control wild type CD8+ T cells using fluorescently labelled VLP-MHC-SIINFEKL. FIG. 5C: Procedure for panning libraries of pMHC VLPs against T cells for detection of cognate pairs, using single-cell RNA sequencing to recover paired TCR-peptide sequences.

FIGS. 6A-6B: Assembly of DogTag-labeled pMHC-VLPs for screening. FIG. 6A. Protein structure of the PP7 coat-protein dimer annotated in magenta with the DogTag insertion site 3D model of the assembled virus-like-particle with DogTag display sites highlighted in magenta (left). Schematic of the assembly of H2kb-DogCatcher with assembled DogTag virus-like-particle (center). Coomassie Blue gel depicting the assembly products of covalently conjugated DogTag VLP with H2kb-DogCatcher (right). FIG. 6B. Specific binding of H2Kb-SIINFEKL (Ovalbumin peptide) DogCatcher conjugated with DogTag VLPs to OT-I mouse CD8+ T cells. Flow analysis shows detection of cognate OT-I T cells but not control wild type CD8+ T cells using fluorescently labelled VLP-MHC-SIINFEKL.

FIGS. 7A-7C: pMHC-VLPs are capable of driving T cell activation and expansion. FIG. 7A: pMHC VLPs are mixed with pooled T cells ex vivo to activate cognate antigen-specific T cells. Activated T cells are enriched via magnetic selection against an activation marker and further expanded in the presence of cytokines. After 7 days, the T cell pool is enriched for pMHC VLP antigen-cognate T cells. The process can be repeated multiple times. FIG. 7B: Addition of pMHC virus-like-particles stimulates CD69 upregulation in OT-I T cells in a concentration dependent manner; activation is independent of CD28 co-stimulation. FIG. 7C: Panel 1: SIINFEKL (Ovalbumin peptide) tetramer detection on OT-I T cells. Panel 2-3: Enrichment of SIINFEKL-specific T cells from a polyclonal pool of 10 million naïve T cell pool on day 8 of the protocol described in 3A.

FIGS. 8A-8B: Encapsulation of antigen peptide barcodes by VLPs. FIG. 8A: An RNA barcode is encapsulated within the VLPs, and consists of a random 15-nt barcode region and adaptors for the 10× Genomics single-cell RNA seq. FIG. 8B: RT-qPCR of 2-fold dilutions of VLP containing a fixed barcode region. Cq=10.67, 11.62, and 12.51 respectively, confirming linear detection of the VLP barcode.

FIGS. 9A-9D: Self-assembly of a bacterial encapsulin protein into a virus-like particle encapsulating cargo protein. FIG. 9A depicts a DogTag peptide inserted into a surface-accessible loop within a T. maritima encapsulin protein after amino acid E135 (EncTM-DogTag). Upon addition of a cargo protein fused to the DogCatcher protein domain, an isopeptide bond forms, linking the cargo covalently with the encapsulin monomer. FIG. 9B: Co-expression of EncTM-DogTag with EGFP linked to DogCatcher (EGFP-DogCatcher) results in covalent conjugation via isopeptide bond to form EncTM-DogTag+EGFP-DogCatcher in E. coli cells. FIG. 9C: Size-exclusion chromatography confirms the proper assembly of EncTM-DogTag monomers into a larger species corresponding to assembled T=1 capsid. In vitro conjugation of EncTM-DogTag with the cargo protein B2M-HLA-DogCatcher confirms the ability to conjugate cargo proteins to pre-assembled EncTM-DogTag capsids. When excess cargo protein was added, the EncTM-DogTag monomer reacted completely via isopeptide bonds to form the larger species EncTM-DogTag+B2M-HLA-DC. FIG. 9D: EncTM-DogTag+B2M-HLA-DC viral-like particles were prepared with H2Kb loaded with SIINFEKL peptide specifically label SIINFEKL-reactive OT-I T cells in a concentration-dependent manner. Fluorescent labeling was accomplished using a secondary antibody against a His-tag contained within the EncTM-DogTag sequence.

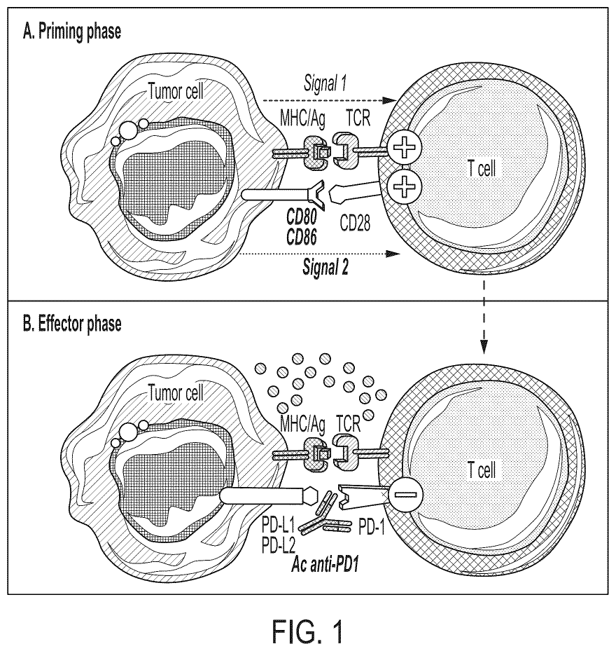

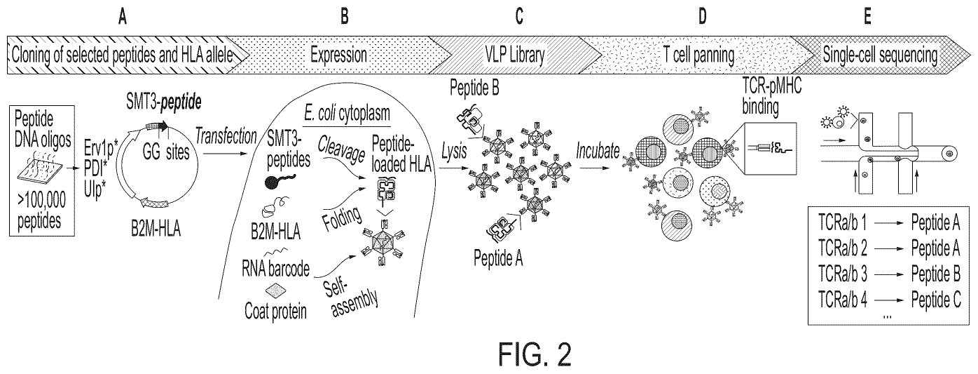

### DETAILED DESCRIPTION

Recognition of which antigen peptide-major histocompatibility complexes (pMHCs) bind to which T cell receptors (TCRs) is crucial for the development of effective therapies for a broad range of pathologies, including cancer, autoimmunity, and pathogenic infections. However effective, high throughput approaches for identifying pMHC-TCR cognate pairs are lacking. The present disclosure is based on various methods and compositions relating to improved identification of pMHC-TCR pairs.

One aspect of the present disclosure provides a method for producing complexes between an antigen peptide and a major histocompatibility complex by providing an Escherichia coli cell under conditions suitable for expression, wherein the E. coli cell comprises one or more sequences encoding a major histocompatibility complex (MHC), one or more sequences encoding one or more heterologous oxidation enzymes, and a sequence encoding an antigen peptide, and then isolating complexes between the antigen peptide and MHC (pMHC) from the E. coli cell.

In some embodiments, the one or more sequences encoding MHC comprise a sequence encoding human leukocyte antigen (HLA) (NCBI Reference Sequence: NM_002116.8; Gene ID: 3105) and a sequence encoding beta-2-microglobulin (B2M) (NCBI Reference Sequence: NM_004048.4; Gene ID: 567). In some embodiments, the sequence encoding HLA has at least 70%, at least 75%, at least 80%, at least 85%, at least 90%, at least 95%, at least 98%, at least 99%, at least 99.5%, or at least 99.9% sequence identity to Gene ID: 3105. In some embodiments, the sequence encoding B2M has at least 70%, at least 75%, at least 80%, at least 85%, at least 90%, at least 95%, at least 98%, at least 99%, at least 99.5%, or at least 99.9% sequence identity to Gene ID: 567. In some embodiments, the one or more sequences encoding one or more heterologous oxidation enzymes comprise a sequence encoding mitochondrial FAD-linked sulfhydryl oxidase (Erv1) and/or a sequence encoding protein disulfide isomerase (PDI). In certain embodiments, the sequence encoding Erv1 is derived from Saccharomyces cerevisiae (NCBI Reference Sequence: NM_001181158.3; Gene ID: 852916). In some embodiments, the sequence encoding Erv1 has at least 70%, at least 75%, at least 80%, at least 85%, at least 90%, at least 95%, at least 98%, at least 99%, at least 99.5%, or at least 99.9% sequence identity to Gene ID: 852916. In certain embodiments, the sequence encoding PDI is derived from S. cerevisiae (NCBI Reference Sequence: NM_001178688.1; Gene ID: 850314) or Homo sapiens (NCBI Reference Sequence: NM_006849.4; Gene ID: 64714). In some embodiments, the sequence encoding PDI has at least 70%, at least 75%, at least 80%, at least 85%, at least 90%, at least 95%, at least 98%, at least 99%, at least 99.5%, or at least 99.9% sequence identity to Gene IDs: 850314 or 64714.

In some embodiments, the sequence encoding an antigen peptide encodes an antigen peptide that is further modified by fusion to a protein label. In certain embodiments, the protein label is ubiquitin-like protein SMT3 (NCBI Reference Sequence: NP_010798.1; Gene ID: 852122). In some embodiments, the sequence encoding an antigen peptide encodes a variant of SMT3, having at least 70%, at least 75%, at least 80%, at least 85%, at least 90%, at least 95%, at least 98%, at least 99%, at least 99.5%, or at least 99.9% sequence identity to Gene ID: 852122. In certain embodiments, the protein label is fused to the N-terminus of the antigen peptide.

In some embodiments, the E. coli cell further comprises one or more sequences encoding a protease that removes the protein label. In certain embodiments, the one or more sequences encoding a protease that removes the protein label comprise ubiquitin-like-specific protease 1 (Ulp1) (NCBI Reference Sequence: NM_001183834.1; Gene ID: 856087). In some embodiments, the one or more sequences encoding a protease encode a variant of Ulp1 having at least 70%, at least 75%, at least 80%, at least 85%, at least 90%, at least 95%, at least 98%, at least 99%, at least 99.5%, or at least 99.9% sequence identity to Gene ID: 856087.

In some embodiments, the antigen peptide is an antigen peptide randomly selected from a library of antigen peptides.

In some embodiments, one or more of the sequences encoding the MHC, the sequences encoding the heterologous oxidation enzymes, the sequence encoding the antigen peptide, and/or the sequence encoding the protease are comprised by one or more plasmids. In some embodiments, one or more of the sequences encoding the MHC, the sequences encoding the heterologous oxidation enzymes, the sequence encoding the antigen peptide, and/or the sequence encoding the protease are integrated into the E. coli genome.

In some embodiments, one or more of the sequences encoding the MHC, the sequences encoding the heterologous oxidation enzymes, the sequence encoding the antigen peptide, and/or the sequence encoding the protease are operably linked to a constitutive promotor. In some embodiments, one or more of the sequences encoding the MHC, the sequences encoding the heterologous oxidation enzymes, the sequence encoding the antigen peptide, and/or the sequence encoding the protease are operably linked to an inducible promotor.

In some embodiments, the method further comprises a step of assaying the isolated pMHC. In certain embodiments, assaying the isolated pMHC comprises purifying the isolated pMHC by affinity chromatography. In further embodiments, isolated pMHC comprises separating the antigen peptide from the pMHC and/or analyzing the antigen peptide by mass spectrometry.

In some embodiments, the isolated pMHC is multimeric.

### Virus-Like Particles (VLPs)

In some aspects, the present disclosure provides a virus-like particle (VLP) or a self-assembling protein nanocompartment (e.g., protein nanocages) conjugated to a pMHC, comprising: a VLP or nanocompartment comprised of: (i) a self-assembling viral or non-viral coat protein, (ii) a major histocompatibility complex (MHC), and (iii) an antigen peptide complexed with the MHC (pMHC), wherein the self-assembling coat protein and MHC are modified by fusion to a binding protein and binding peptide, respectively, and wherein the binding protein conjugates to the binding peptide.

As used herein, virus-like particles (VLPs) refer to self-assembled protein nanostructures composed of multiple copies of one or more coat proteins, which may be envelope or structural coat proteins. These particles resemble their corresponding natural viruses in structure but lack the genomic cargo necessary for replication, and are typically stable and biocompatible (Zhao et al., “Engineering the PP7 Virus Capsid as a Peptide Display Platform.” ACS Nano. 2019; 13(4):4443-4454). VLPs derived from RNA bacteriophages (e.g., PP7 or MS2) are particularly amenable to high-yield expression and assembly in different systems, including bacteria, yeast, insect cells, and mammalian cells; many of these characteristics derive from their polyvalency (e.g., the ability of VLPs to present multiple copies of functional peptides or protein domains to potential binding agents, cells, and tissues). In some implementations of the present disclosure, the VLPs are comprised of capsids formed by coat proteins derived from RNA bacteriophages (e.g., PP7 or MS2), which have been shown to be physically and thermally stable (Caldeira and Peabody, “Stability and assembly in vitro of bacteriophage PP7 virus-like particles.” J Nanobiotechnology. 2007; 5:10).

As used herein, the “virus-like particles” (i.e., VLPs) also encompass protein nanocages (e.g., self-assembling protein nanocompartments), which are biocompatible and can be produced in bacteria, allowing large-scale production and protein engineering. Protein nanocages are complex macromolecular aggregates, generated from the precise self-assembly of repeated protein subunits. As molecular components for nanomedical devices for the delivery of antigens and therapeutic molecules, self-assembled protein cages display numerous advantages, including the possibility of rational and geometrically defined surface modifications with molecular precision. Furthermore, protein cages are capable of carrying heterologous cargos via a range of native and heterologous encapsidation strategies (Steinmetz et al., “Protein cages and virus-like particles: from fundamental insight to biomimetic therapeutics.” Biomater Sci. 2020; 8(10):2771-2777). Among naturally occurring protein nanocages, encapsulins have emerged as an alternative engineering platform for applications in medicine, catalysis, and nanotechnology. Encapsulins are self-assembling icosahedral protein compartments composed of a single type of shell protomer possessing the HK97 phage-like fold. Encapsulins can assemble into T1 (60 subunits, ca. 24 nm), T3 (180 subunits, ca. 32 nm), and T4 (240 subunits, ca. 42 nm) shells and are widely distributed throughout the bacterial and archaeal domains (Kwon and Giessen, “Engineered Protein Nanocages for Concurrent RNA and Protein Packaging In Vivo.” ACS Synth Biol. 2022; 11(10):3504-3515; Michel-Souzy et al., “Introduction of Surface Loops as a Tool for Encapsulin Functionalization.” Biomacromolecules. 2021; 22(12):5234-5242). A key feature of encapsulins is the ability to selectively encapsulate dedicated cargo proteins in vivo. In some implementations of the present disclosure, the VLPs are comprised of capsids formed by encapsulin proteins, optionally wherein the encapsulin protein is a bacterial encapsulin protein derived from T. maritima.

The encapsulin proteins used in the present disclosure may form capsids in any geometry, including T=1 using 180 copies, T=3 using 540 copies, or T=4 with 720 copies. In some embodiments, the encapsulin protein is derived from T. maritima. In some embodiments, the self-assembling T. maritima encapsulin protein has the following amino acid sequence (Protein Database (PDB) Reference Sequence: Q9WZP2):

(SEQ ID NO: 11)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKF.

In some embodiments, the self-assembling coat protein is a variant of the T. maritima encapsulin protein set forth in SEQ ID NO: 11 or a variant of the T. maritima encapsulin protein having at least 80%, at least 85%, at least 90%, at least 95%, at least 98%, at least 99%, at least 99.5%, or at least 99.9% identity to the sequence set forth in SEQ ID NO: 11. In some embodiments, the variant of the encapsulin protein comprises a DogTag peptide sequence (e.g., SEQ ID NO: 9), or a peptide having at least 80%, at least 85%, at least 90%, at least 95%, at least 98%, at least 99%, at least 99.5%, or at least 99.9% identity to SEQ ID NO: 9, wherein the DogTag peptide is inserted after amino acid position corresponding to E135 with reference to SEQ ID NO: 11. In some embodiments, the variant of the encapsulin protein comprises one or more linker sequences, wherein optionally the linker sequence is GGGGS (SEQ ID NO: 13) or SG. In some embodiments, the linker sequence has at least 80%, at least 85%, at least 90%, at least 95%, at least 98%, at least 99%, at least 99.5%, at least 99.8%, or at least 99.9% sequence identity to the sequence set forth in SEQ ID NO: 13. In some embodiments, the linker sequence is about 2 amino acids, about 3 amino acids, about 4 amino acids, about 5 amino acids, about 10 amino acids, about 15 amino acids, about 20 amino acids, or longer in length. A linker sequence as described herein may be attached to the N-terminus, C-terminus, and/or at an internal site of a protein of interest. In some embodiments, the proteins described herein (e.g., a self-assembling coat protein) is modified to comprise a protein tag (e.g., a 6× His tag, e.g., HHHHHH), which may be attached to the N-terminus, C-terminus, and/or at an internal site of a protein of interest. In some embodiments, the variant of the encapsulin protein has the following amino acid sequence:

(SEQ ID NO: 12)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEGGGGSDIPATYEFTD
GKHYITNEPIPPKGGGGSERKIECGSTPKDLLEAIVRALSIFSKDGIEGP
YTLVINTDRWINFLKEEAGHYPLEKRVEECLRGGKIITTPRIEDALVVSE
RGGDFKLILGQDLSIGYEDREKDAVRLFITETFTFQVVNPEALILLKFSG
HHHHHH.

In some embodiments, the variant of the encapsulin protein is a variant having at least 80%, at least 85%, at least 90%, at least 95%, at least 98%, at least 99%, at least 99.5%, or at least 99.9% sequence identity to the sequence set forth in SEQ ID NO: 12.

In some embodiments, the self-assembling coat protein is a MS2 or PP7 bacteriophage coat protein.

In some embodiments, a MS2 bacteriophage coat protein is a self-assembling viral coat protein having the following amino acid sequence (NCBI Reference Sequence: YP_009640125.1):

(SEQ ID NO: 1)
MASNFTQFVLVDNGGTGDVTVAPSNFANGVAEWISSNSRSQAYKVTCSVR
QSSAQNRKYTIKVEVPKVATQTVGGVELPVAAWRSYLNMELTIPIFATNS
DCELIVKAMQGLLKDGNPIPSAIAANSGIY.

In some embodiments, the self-assembling viral coat protein is a variant of a MS2 bacteriophage coat protein having at least 80%, at least 85%, at least 90%, at least 95%, at least 98%, at least 99%, at least 99.5%, or at least 99.9% sequence identity to the sequence set forth in SEQ ID NO: 1.

In some embodiments, a PP7 bacteriophage coat protein is a self-assembling viral coat protein having the following amino acid sequence (NCBI Reference Sequence: NP_042305.1):

(SEQ ID NO: 2)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVATSQVEDLVVNLVPLGR.

In some embodiments, the self-assembling viral coat protein is a variant of a PP7 bacteriophage coat protein having at least 80%, at least 85%, at least 90%, at least 95%, at least 98%, at least 99%, at least 99.5%, or at least 99.9% sequence identity to the sequence set forth in SEQ ID NO: 2.

In some embodiments, the binding peptide and the binding protein conjugate through a covalent bond.

In some embodiments, the binding peptide is SpyTag and the binding protein is SpyCatcher (see, e.g., Keeble, et al. Proc Natl Acad Sci USA. 2019; 116(52):26523-33). In some embodiments, the binding peptide is a SpyTag peptide having the following amino acid sequence, generally referred to in the art as “SpyTag”: AHIVMVDAYKPTK (SEQ ID NO: 3). In some embodiments, the binding peptide is a SpyTag peptide having the following amino acid sequence, generally referred to in the art as “SpyTag002”: VPTIVMVDAYKRYK (SEQ ID NO: 4). In some embodiments, the binding peptide is a SpyTag peptide having the following amino acid sequence, generally referred to in the art as “SpyTag003”: RGVPHIVMVDAYKRYK (SEQ ID NO: 5). In some embodiments, the binding peptide is a variant of SpyTag having at least 80%, at least 85%, at least 90%, at least 95%, at least 98%, at least 99%, at least 99.5%, or at least 99.9% sequence identity to any one of the sequences set forth in SEQ ID NOs: 3-5.

In some embodiments, the binding protein is a SpyCatcher protein having the following amino acid sequence, generally referred to in the art as “SpyCatcher”:

(SEQ ID NO: 6)
VDTLSGLSSEQGQSGDMTIEEDSATHIKFSKRDEDGKELAGATMELRDSS
GKTISTWISDGQVKDFYLYPGKYTFVETAAPDGYEVATAITFTVNEQGQV
TVNGKATKGDAHI.

In some embodiments, the binding protein is a SpyCatcher protein having the following amino acid sequence, generally referred to in the art as “SpyCatcher002”:

(SEQ ID NO: 7)
VTTLSGLSGEQGPSGDMTTEEDSATHIKFSKRDEDGRELAGATMELRDSS
GKTISTWISDGHVKDFYLYPGKYTFVETAAPDGYEVATAITFTVNEQGQV
TVNGEATKGDAHT.

In some embodiments, the binding protein is a SpyCatcher protein having the following amino acid sequence, generally referred to in the art as “SpyCatcher003”:

(SEQ ID NO: 8)
VTTLSGLSGEQGPSGDMTTEEDSATHIKFSKRDEDGRELAGATMELRDSS
GKTISTWISDGHVKDFYLYPGKYTFVETAAPDGYEVATPIEFTVNEDGQV
TVDGEATEGDAHT.

In some embodiments, the binding protein is a variant of SpyCatcher having at least 80%, at least 85%, at least 90%, at least 95%, at least 98%, at least 99%, at least 99.5%, or at least 99.9% sequence identity to the sequence set forth in any one of SEQ ID NOs: 6-8.

In some embodiments, the binding peptide is that set forth in SEQ ID NO: 3 or a variant thereof, and the binding protein is that set forth in SEQ ID NO: 6 or a variant thereof. In some embodiments, the binding peptide is that set forth in SEQ ID NO: 4 or a variant thereof, and the binding protein is that set forth in SEQ ID NO: 7 or a variant thereof. In some embodiments, the binding peptide is that set forth in SEQ ID NO: 5 or a variant thereof, and the binding protein is that set forth in SEQ ID NO: 8 or a variant thereof.

In some embodiments, the binding peptide is DogTag and the binding protein is DogCatcher (see, e.g., Keeble, et al. Cell Chem Biol. 2022; 29(2):339-350.e10). In some embodiments, the binding peptide is a DogTag peptide having the following amino acid sequence: DIPATYEFTDGKHYITNEPIPPK (SEQ ID NO: 9). The binding peptide (e.g., DogTag or SpyTag) may be attached to the N-terminus, the C-terminus, or at an internal site of a protein of interest. In some embodiments, the binding peptide is a variant of DogTag having at least 80%, at least 85%, at least 90%, at least 95%, at least 98%, at least 99%, at least 99.5%, or at least 99.9% sequence identity to the sequence set forth in SEQ ID NO: 9. In some embodiments, the binding protein is a DogCatcher protein having the following amino acid sequence:

(SEQ ID NO: 10)
MKLGEIEFIKVDKTDKKPLRGAVFSLQKQHPDYPDIYGAIDQNGTYQDVR
TGEDGKLTFTNLSDGKYRLIENSEPPGYKPVQNKPIVSFRIVDGEVRDVT
SIVPQ.

In some embodiments, the binding protein is a variant of DogCatcher having at least 80%, at least 85%, at least 90%, at least 95%, at least 98%, at least 99%, at least 99.5%, or at least 99.9% sequence identity to the sequence set forth in SEQ ID NO: 10.

In some embodiments, the binding peptide is that set forth in SEQ ID NO: 9 or a variant thereof, and the binding protein is that set forth in SEQ ID NO: 10 or a variant thereof.

In some embodiments, beta-2-microglobulin (B2M) is presented as an N-terminal fusion to the coat protein, providing an anchor for non-covalent pMHC complex assembly upon protein folding. In some embodiments, DogTag, or a variant thereof, is inserted into a dimeric PP7 virus-like particle coat protein within the surface-accessible loop, and pMHC is C-terminally fused to DogCatcher or a variant thereof. In some embodiments, DogTag, or a variant thereof, is inserted into a dimeric MS2 virus-like particle coat protein within the surface-accessible A loop, pMHC is C-terminally fused to DogCatcher or a variant thereof.

Sequence identity, including determination of sequence complementarity for nucleic acids or amino acid sequences, may be determined by sequence comparison and alignment algorithms known in the art. To determine the percent identity of two amino acid sequences, the sequences are aligned for optimal comparison purposes (e.g., gaps can be introduced in the first sequence or second sequence for optimal alignment). The amino acids at corresponding nucleotide positions are then compared. When a position in the first sequence is occupied by the same amino acid as the corresponding position in the second sequence, then the molecules are identical at that position. In some embodiments, the percent identity between the two sequences is a function of the number of identical positions shared by the sequences (e.g., % homology=# of identical positions/total # of positions×100), optionally penalizing the score for the number of gaps introduced and/or length of gaps introduced.

As described herein, alignments between sequences of nucleic acids or polypeptides are performed using any of a variety of publicly or commercially available Multiple Sequence Alignment Programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two proteins or peptides. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid. Table 6 provides examples of variant amino acids.

In some embodiments, the VLP comprises up to 90 sites for conjugation with pMHC. In some embodiments, the VLP is conjugated to up to 90 pMHCs. In some embodiments, the VLP comprises up to 180 sites for conjugation with pMHC. In some embodiments, the VLP is conjugated to up to 180 pMHCs.

In some embodiments, the antigen peptides of pMHCs complexed with the VLP are the same. In some embodiments, the antigen peptides of pMHCs complexed with the VLP are different.

In some embodiments, the pMHCs are multimeric.

In another aspect, the present disclosure provides a method for producing VLPs conjugated to pMHC, comprising providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide, and then isolating VLPs conjugated to complexes between the antigen peptide and MHC (pMHC) from the E. coli cell.

In some embodiments, the one or more sequences encoding MHC comprise a sequence encoding human leukocyte antigen (HLA) and a sequence encoding beta-2-microglobulin (B2M).

In some embodiments, the one or more sequences encoding one or more heterologous oxidation enzymes comprise a sequence encoding mitochondrial FAD-linked sulfhydryl oxidase (Erv1) and/or a sequence encoding protein disulfide isomerase (PDI). In certain embodiments, the sequence encoding Erv1 is derived from S. cerevisiae. In certain embodiments, the sequence encoding PDI is derived from S. cerevisiae or H. sapiens.

In some embodiments, the sequence encoding an antigen peptide encodes an antigen peptide that is further modified by fusion to a protein label. In certain embodiments, the protein label is ubiquitin-like protein SMT3. In certain embodiments, the protein label is fused to the N-terminus of the antigen peptide.

In some embodiments, the E. coli cell further comprises one or more sequences encoding a protease that removes the protein label. In certain embodiments, the one or more sequences encoding a protease that removes the protein label comprise ubiquitin-like-specific protease 1 (Ulp1).

In some embodiments, the antigen peptide is an antigen peptide randomly selected from a library of antigen peptides.

In some embodiments, the self-assembling coat protein is a MS2 or PP7 bacteriophage coat protein.

In some embodiments, the VLPs each comprise up to 180 sites for conjugation with pMHC.

In some embodiments, the pMHCs are multimeric.

In some embodiments, the binding peptide and the binding protein conjugate through a covalent bond. In some embodiments, the binding peptide is SpyTag, or a variant thereof, and the binding protein is SpyCatcher or a variant thereof. In some embodiments, the binding peptide is DogTag, or a variant thereof, and the binding protein is DogCatcher or a variant thereof. In some embodiments, the binding peptide is fused to the C-terminus of the MHC and/or the binding protein is fused to the C-terminus of the self-assembling coat protein.

In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are comprised by one or more plasmids. In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are integrated into the E. coli genome.

In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are operably linked to a constitutive promotor. In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are operably linked to an inducible promotor.

In another aspect, the present disclosure provides a method for identifying pMHC-TCR pairs, comprising providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide, isolating VLPs conjugated to complexes between the antigen peptide and MHC (pMHC) from the E. coli cell, contacting the isolated VLPs with a population of T cells, sequencing the population of T cells, and then determining pMHC-T cell receptor (TCR) cognate pairs by identifying TCR-encoding sequences and antigen peptide-encoding sequences comprised by each T cell within the population of T cells.

In some embodiments, the one or more sequences encoding MHC comprise a sequence encoding human leukocyte antigen (HLA) and a sequence encoding beta-2-microglobulin (B2M).

In some embodiments, the one or more sequences encoding one or more heterologous oxidation enzymes comprise a sequence encoding mitochondrial FAD-linked sulfhydryl oxidase (Erv1) and/or a sequence encoding protein disulfide isomerase (PDI). In certain embodiments, the sequence encoding Erv1 is derived from S. cerevisiae. In certain embodiments, the sequence encoding PDI is derived from S. cerevisiae or H. sapiens.

In some embodiments, the sequence encoding an antigen peptide encodes an antigen peptide that is further modified by fusion to a protein label. In certain embodiments, the protein label is ubiquitin-like protein SMT3. In certain embodiments, the protein label is fused to the N-terminus of the antigen peptide.

In some embodiments, the E. coli cell further comprises one or more sequences encoding a protease that removes the protein label. In certain embodiments, the one or more sequences encoding a protease that removes the protein label comprise ubiquitin-like-specific protease 1 (Ulp1).

In some embodiments, the antigen peptide is an antigen peptide randomly selected from a library of antigen peptides.

In some embodiments, the self-assembling coat protein is a MS2 or PP7 bacteriophage coat protein, or a variant thereof.

In some embodiments, the VLPs each comprise up to 180 sites for conjugation with pMHC.

In some embodiments, the pMHCs are multimeric.

In some embodiments, the binding peptide and the binding protein conjugate through a covalent bond. In some embodiments, the binding peptide is SpyTag, or a variant thereof, and the binding protein is SpyCatcher or a variant thereof. In some embodiments, the binding peptide is DogTag, or a variant thereof, and the binding protein is DogCatcher or a variant thereof. In some embodiments, the binding peptide is fused to the C-terminus of the MHC and/or the binding protein is fused to the C-terminus of the self-assembling coat protein.

In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are comprised by one or more plasmids. In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are integrated into the E. coli genome.

In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are operably linked to a constitutive promotor. In some embodiments, one or more of the sequences encoding the MHC, the sequences encoding the heterologous oxidation enzymes, the sequence encoding the antigen peptide, the sequence encoding the protease, and/or the sequence encoding the self-assembling coat protein are operably linked to an inducible promotor.

In some embodiments, the sequence encoding the antigen peptide further encodes a barcode sequence. In some embodiments, the barcode sequence is a DNA or RNA sequence. In some embodiments, the barcode sequence is encapsulated by the virus-like particles (VLPs). In some embodiments, the step of determining pMHC-T cell receptor (TCR) cognate pairs comprises identifying TCR-encoding sequences and barcode sequences comprised by each T cell within the population of T cells.

In some embodiments, the step of sequencing the population of T cells comprises single cell RNA sequencing (RNA-seq).

In some embodiments, the antigen peptide is an antigen peptide from a cancer cell. In some embodiments, the antigen peptide is an antigen peptide from a bacterial cell, parasite cell, or virus.

In some embodiments, the antigen peptide is an antigen peptide belonging to no known pMHC-TCR cognate pair.

### EXAMPLES

### Example 1

T cells play a central role in cancer immunotherapy but determining their antigen-specificity remains extremely challenging experimentally and not yet feasible computationally (Peters, Nielsen, and Sette 2020). Next-generation sequencing has made it possible to directly determine the primary sequence of the TCR, but the structural diversity of the T cell receptor and the polymorphism of MHC genes have precluded direct prediction of the peptide-MHC-TCR complex (pMHC-TCR). Moreover, the number of directly validated pMHC-TCR interactions (in the thousands) is miniscule compared to the size of the human TCR repertoire (in the high millions based on recent measurements of the beta chain alone, but multiplicatively more based on random combinations of both chains (Qi et al. 2014)). There is a critical need for scalable methods for discovering pMHC-TCR interactions that can then be used to train generalized, predictive models of pMHC-TCR binding using machine learning. These predictive models can then be used to determine if TCRs found in patients are reactive to tumor antigens predicted from sequence data.

Existing approaches are inadequate for experimentally discovering matched TCRs and cognate peptides and are limited to (1) searching for peptides that match a single TCR (one TCR to many peptides), or (2) searching for TCRs that bind one peptide (one peptide to many TCRs). To date, only 105 peptides have been mapped to at least 50 TCRs per peptide (Chronister et al. 2021), which is the minimum number of TCRs needed to classify TCRs that bind a particular pMHC. While DNA-barcoded pMHC (multimers) have been used to detect antigen-specific T cells (Bentzen et al. 2016) they are challenging to produce since they must be assembled individually per chemically synthesized peptide and per recombinant HLA allele protein. Other methods use yeast surface-display of a pMHC library to identify peptides that bind purified TCR-tetramers, but are limited to searching against a small number of TCRs expressed and purified in vitro (Gee et al. 2017). At this time, there is no flexible, high-throughput method for mapping a pool of T cells to a library of peptides (many TCRs to many pMHCs).

### Example 2

A set of methods have been developed for producing natively folded peptide-loaded MHC class I complexes in the E. coli cytoplasm. The method is compatible with standard plasmid-based protein library production techniques, which makes it possible for us to generate libraries of greater than 10⁹ unique pMHC complexes in a pooled format. By including a multivalent scaffold for pMHC assembly in these expression constructs, one can produce highly diverse libraries of uniform (same peptide sequence per multimer) pMHC multimers (i.e., tetramers, pentamers, etc.) that are capable of binding to T cells through multivalent interaction with a cognate TCR.

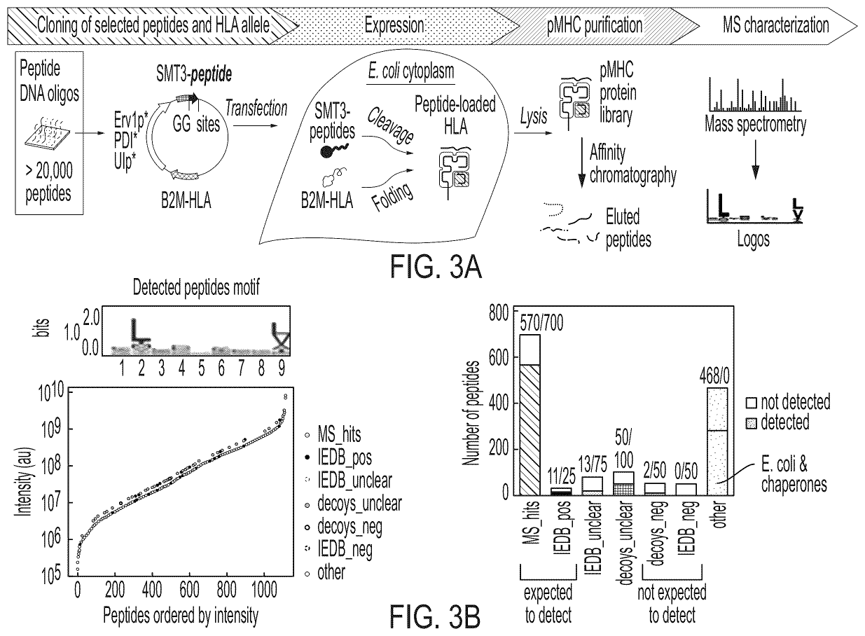

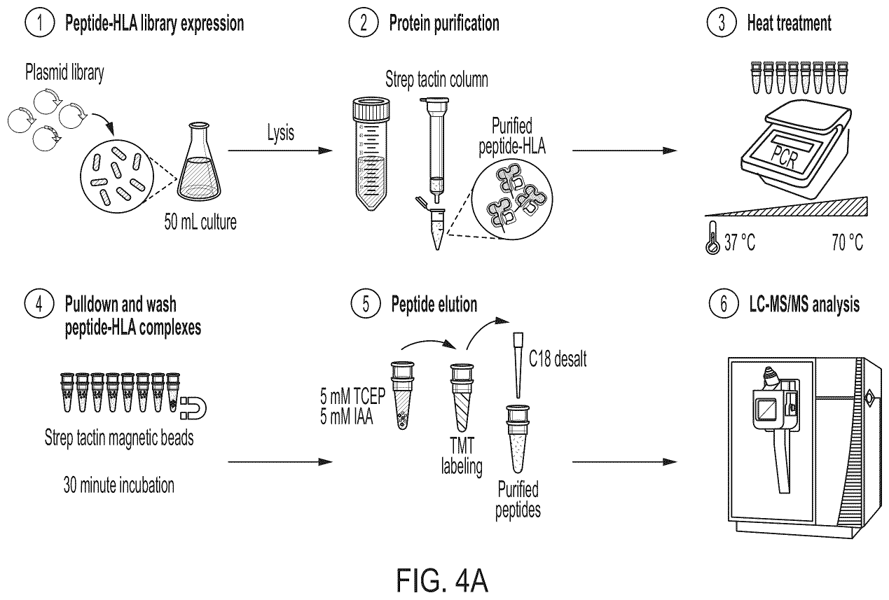

To produce a pMHC library, peptide-encoding oligos are cloned into a plasmid encoding the following transcripts: (i) single-chain disulfide-stabilized B2M-HLA with Strep-II tag for pulldown; (ii) a multicistronic transcript for oxidative folding chaperones Erv1p and protein disulfide isomerase (PDI), and SUMO protease (Ulp1); (iii) SUMO (SMT3) followed by a Golden-Gate cloning site for insertion of peptide-coding oligos during library cloning (SMT3-peptide) (FIG. 3A). Following transformation into E. coli and translation, the SMT3-peptide is cleaved by Ulp1 to release an epitope peptide that is loaded into the peptide-binding groove of single-chain B2M-HLA, generating a user-defined peptide-HLA complex.

To demonstrate that the HLA-bound peptides follow the same rules as HLA-bound peptides identified from mammalian cells, a library of 1000 peptides with known binding properties to HLA-A0201 was designed, cloned, and produced. After isolating the pMHCs from E. coli and eluting bound peptides, peptides were identified by mass spectrometry. The recovered peptides corresponded with >80% of known A*02:01 binders and 0% of known non-binders (FIG. 3B), establishing the capability to generate pMHC complexes loaded with user-defined peptide libraries.

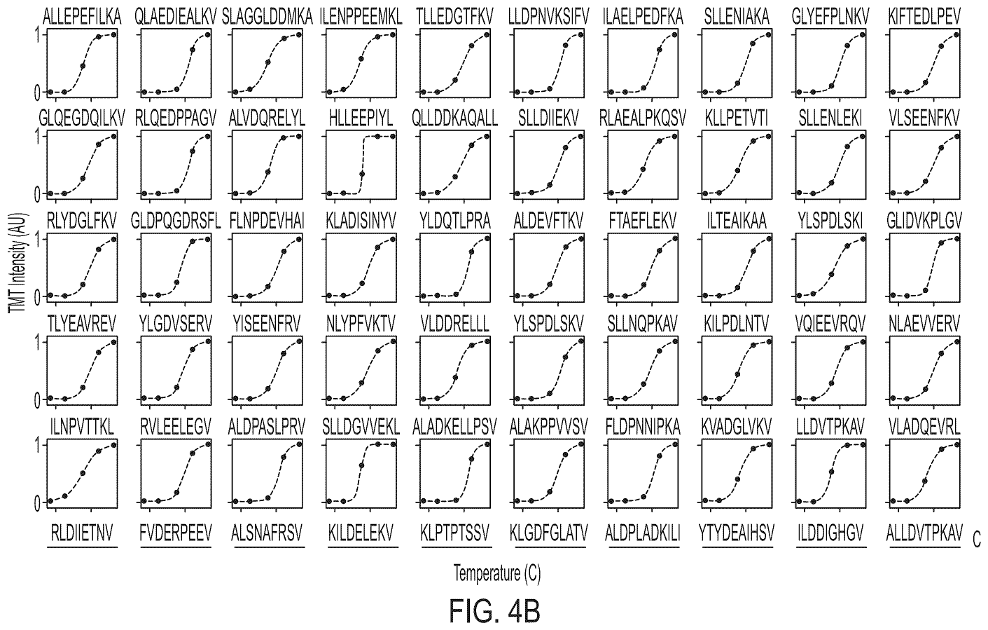

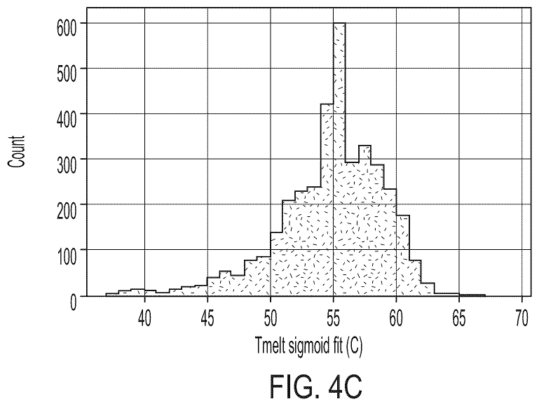

To further demonstrate that HLA-bound peptides represent the natural HLA-peptide binding mode, the thermostability of peptides in the HLA-A2 binding pocket was measured for a library of more than 10,000 peptides. To measure thermostability of peptide-HLA binding, a library of peptides was designed, cloned, and produced, and after isolating the pMHCs from E. coli, the sample was divided into 5 wells and subject to a heat treatment across a gradient from 37° C. to 73° C. for 10 minutes (FIG. 4A). Heat-eluted peptides were collected, labeled with TMT labeling reagents, and quantitative mass-spectrometry was used to determine the relative quantity of each identified peptides eluted at each temperature. After quality control more than 5,000 peptide melting curves were determined (FIG. 4B). Melting curves were fit to a logistic function, and the Tm (½) distribution of the peptides corresponded to the known biophysical properties of HLA-peptide complexes (˜40° C. <Tm <65° C.) (FIG. 4C).

Further methods have been developed for producing RNA-barcoded pMHC multimers that can be decoded using single-cell RNA sequencing methods. Among these, a multivalent virus-like-particle (VLP) bound with pMHC has been developed in E. coli that encapsulates an RNA barcode encoding the peptide identity. The ability of these VLPs to bind specifically to cognate T cells with the sensitivity of classic tetramers has been demonstrated. Single-cell RNA sequencing of VLP-bound T cells can be used to simultaneously recover T-cell receptor V(D)J sequences and cognate antigen sequences. The methods are very accessible to a standard laboratory with molecular biology expertise, and VLP yield can be scaled inexpensively, resulting in a flexible platform for DNA-programmable interrogation of T cell specificity at unprecedented throughput and scale.

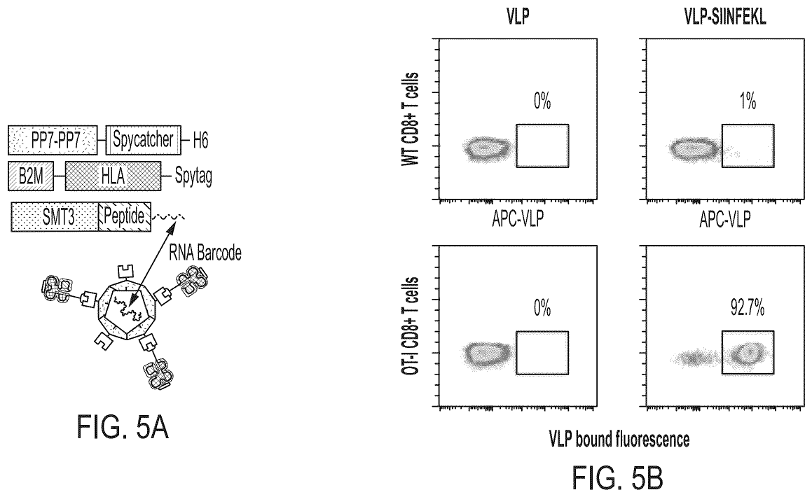

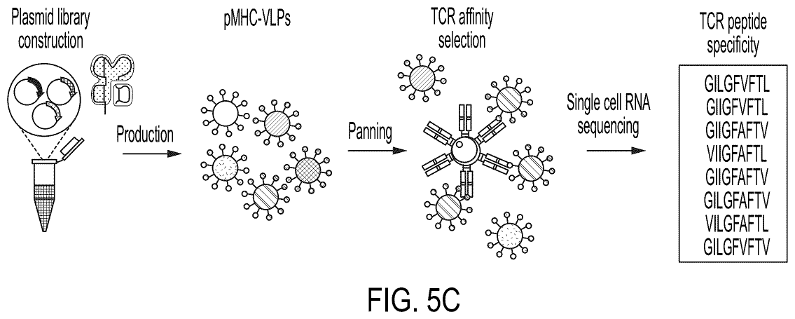

A system for the production of DNA-programmed multimeric pMHC complexes was developed with (1) high display valency to detect low-affinity TCRs; (2) RNA barcoding capacity to encode the displayed peptide; (3) high expression yields in E. coli to produce concentrated complexes for T cell detection. To achieve this, a virus-like-particle based on the coat-protein of the PP7 Levivirus is used, which spontaneously assembles in the E. coli cytoplasm, encapsulates mRNA, and provides 90 sites for pMHC display (vs. ˜1-3 for M13 phage display). A protein construct containing the PP7 coat-protein dimer fused to SpyCatcher was developed. Co-expression of SpyTag-fused HLA-B2M results in covalent immobilization of pMHC on the surface of the VLPs (FIG. 5A).

The applications for this system in T cell antigen discovery are numerous and include (1) identification of tumor-specific TCRs and their cognate peptides, (2) identification of TCRs in infectious disease and autoimmunity, (3) deep-profiling of the specificity of a single or set of TCRs (FIGS. 5B-5C). Finally, most intriguing is the capability to collect large-scale datasets pairing TCRs and cognate peptides, constructing a dataset that can be used to develop predictive computational models of TCR-peptide specificity.

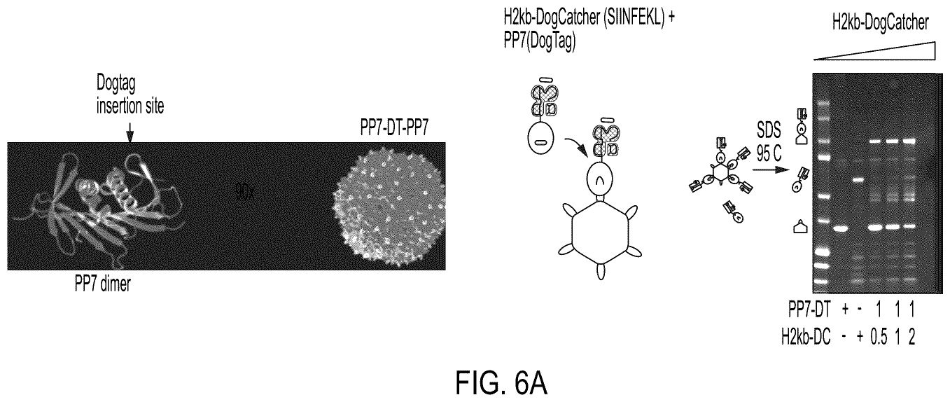

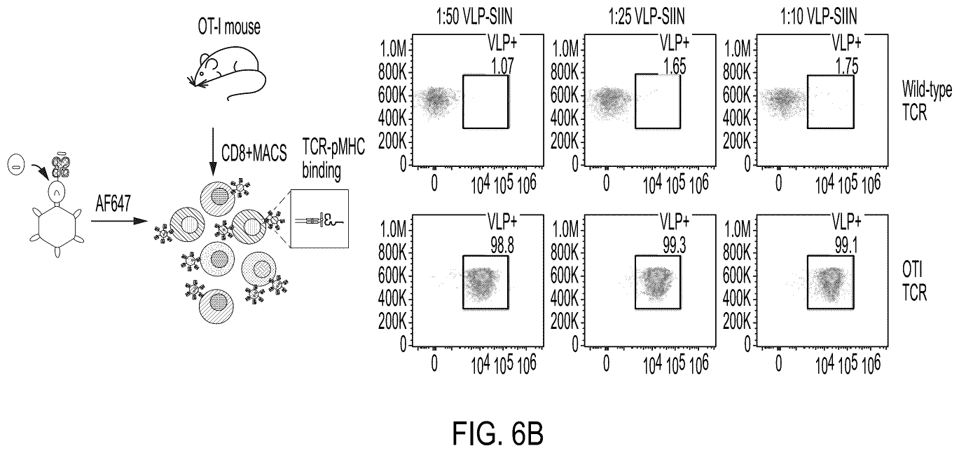

In addition to VLPs based on SpyTag fusions, a virus-like-particle based on the coat-protein of the PP7 or MS2 Levivirus is modified to insert the DogTag sequence into a surface accessible loop (FIG. 6A), and a Dogcatcher domain is appended to the C-terminus of a single-chain B2M-MHC protein. Co-expression of Dogcatcher-fused HLA-B2M results in covalent immobilization of pMHC on the surface of the DogTag-modified VLPs. Specific binding of H2Kb-Dogcatcher-SIINFEKL DogTag-VLPs to OT-I mouse CD8+ T cells was further examined. Flow analysis demonstrated detection of cognate OT-I T cells, but not control wild type CD8+ T cells using fluorescently labelled VLP-MHC-SIINFEKL (FIG. 6B).

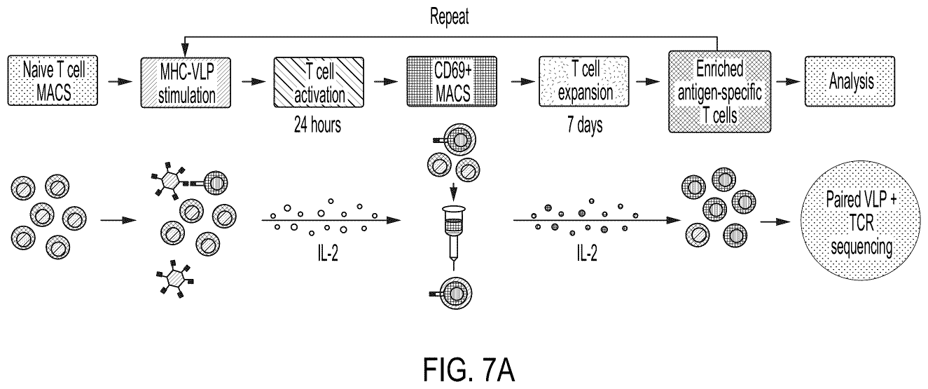

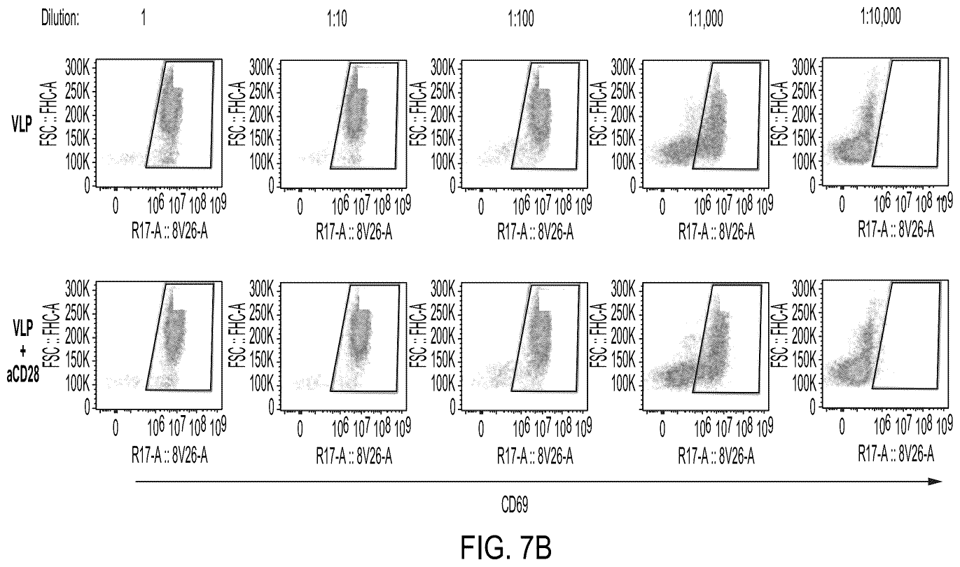

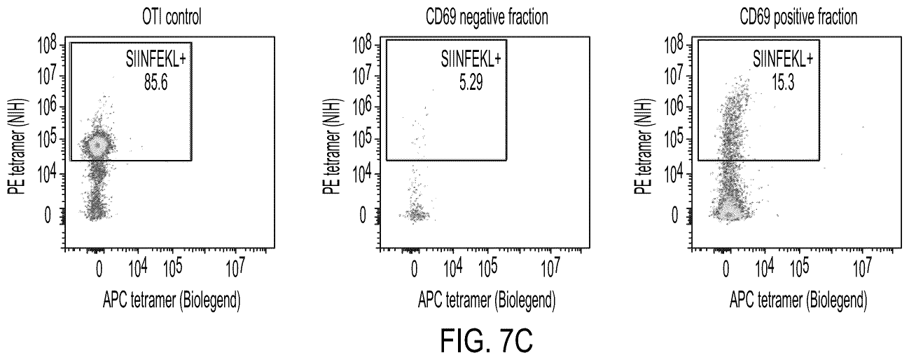

This method also enables the activation, expansion, and isolation of antigen-specific T cells via incubation with pMHC-VLPs; multivalent engagement of the TCR with up to 180 copies of pMHC on the VLP surface is sufficient to drive T cell activation in the absence of costimulatory signals (FIGS. 7A-7C). This method can be used to select for a library of antigen specific T cells from a polyclonal pool of T cells for numerous applications, including T cell discovery or enrichment of antigen-specific T cells for adoptive cell therapy.

### Example 3

The invention relates to; (1) production of properly folded, peptide loaded pMHC complexes in the E. coli cytoplasm; (2) multimerization of pMHC complexes in the E. coli cytoplasm using a protein scaffold; (3) RNA (or DNA) barcoding of pMHC multimers in the E. coli cytoplasm via an RNA binding protein domain or via encapsulation of RNA in a self-assembled protein nanoparticle (e.g., a virus-like particle or a self-assembling protein nanocompartment). The assembly of (1), (2), and (3) is defined by a plasmid encoding multiple protein constructs that direct the assembly of the final composition, which is subsequently purified for final use. The primary use of this invention is the detection of antigen-specific T cells, much like pMHC tetramers are currently used.

The methods described throughout the invention are relevant to Class I MHC complexes, as well as Class II MHC complexes with only minimal modification.

Methods for producing pMHC complexes utilize a plasmid containing (a) HLA protein and B2M, (b) protein chaperones that support oxidative folding in the E. coli cytoplasm, (c) a system for producing epitope-length peptides with native N-termini.

In one implementation, (b) consists of the proteins yeast Erv1p and human PDI (known under the name CyDisCo (Gaciarz et al, 2017).

In one implementation, (c) consists of a SUMO domain fused to the epitope peptide (SUMO-peptide), expressed along with SUMO-protease, which cleaves the SUMO-peptide to release the epitope-length peptide.

In another implementation, (c) consists of a peptide fused to Enterokinase recognition sequence, expressed along with the Enterokinase.

By including (a) a protein domain that self-assembles into a multimer (i.e., trimer, tetramer, pentamer, etc.), attached to the MHC protein directly (on the same polypeptide chain) or through covalent or non-covalent association, uniform (one peptide sequence) multimers are produced within the E. coli cytoplasm (each E. coli cell produces many pMHC multimers loaded with the same peptide).

In one implementation, (a) consists of a naturally multimeric protein domain; examples include streptavidin, a (tetrameric, pentameric, hexameric, etc.) coiled-coil, and the heptameric human protein C4bp and related domains.

In another implementation, (a) consists of a coat-protein that self-assembles into a nanoparticle (e.g., a self-assembling protein nanocompartment) or virus-like particle; examples include the coat protein from the MS2 or PP7 RNA phage, which self-assembled into a 180-mer capsid, or ferritin, which self-assembles into a nanoparticle.

In one implementation, covalent attachment of pMHC to the multimeric protein domain is achieved via fusion of the SpyCatcher and SpyTag domains, which form a spontaneous covalent bond, to either component. In another implementation, covalent attachment of pMHC to the multimeric protein domain is achieved via fusion of the DogCatcher and DogTag domains, which similarly form a spontaneous covalent bond, to either component.

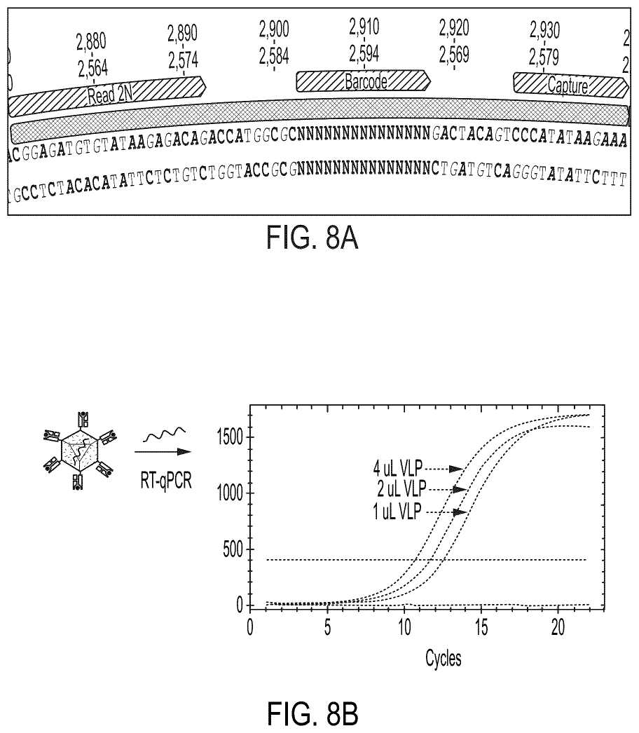

By including (a) a protein domain that binds or encapsulates an RNA sequence, attached to the MHC multimer either directly or through covalent or non-covalent association, this method can produce pMHC multimers that carry an RNA species encoding the identity of the loaded peptide (phenotype to genotype connection, as in phage display or mRNA display). When these multimers are used downstream to detect cognate T cells, the RNA barcode can be decoded via sequencing to reveal the peptide antigen that binds a given T cell (FIGS. 8A-8B).

In one implementation, (a) consists of a protein domain that binds to a specific RNA sequence, such as the MS2 coat protein dimer.

In another implementation, (a) consists of a nanoparticle that encapsulates RNA either specifically or non-specifically, such as an engineered dimeric PP7 or MS2 virus-like particle with a DogTag sequence inserted into a surface-accessible loop of the coat protein.

In another implementation, the scaffold consists of an RNA transcript itself containing one or more binding sites for an RNA protein binding domain linked to pMHC (for instance, an RNA scaffold consisting of multiple MS2-binding hairpins).

In one implementation, the RNA barcode consists of a sequence adaptor that is compatible with the 10× Genomics single-cell RNA sequencing workflow (i.e., feature barcoding).

In additional implementations, the pMHC complex is directly linked to an RNA aptamer binding domain, and 1, 2, 3, 4, 5, 6, 7, 8, 9, or 10 copies of the RNA aptamer are contained in the 3′ UTR of the peptide-encoding RNA, providing a platform for multimeric assembly of the pMHC complex. In some implementations, the RNA aptamer binding domain is the MS2 coat-protein dimer and the RNA aptamer sequence is the MS2 operator hairpin. In some implementations, the RNA aptamer binding domain is the AN peptide or peptide variant and the RNA aptamer sequence is the λN-specific boxB site.

The invention further relates to a method for producing multimeric pMHC linked non-covalently to the peptide-encoding RNA, comprising providing an E. coli cell under conditions suitable for expression, wherein the E. coli cell comprises a sequence encoding one or more antigen peptides and containing one or more RNA aptamer sequences in the 3′ UTR, one or more sequences encoding a major histocompatibility complex (MHC) modified by fusion with an RNA aptamer binding domain capable of conjugating to the RNA aptamers, one or more sequences encoding one or more heterologous oxidation enzymes, and then isolating pMHC multimeric complexes conjugated to the antigen peptide and the peptide-encoding RNA from the E. coli cell.

Together these techniques permit production of properly folded MHC complexes in the cytoplasm of E. coli; production of peptide-loaded HLA class I in the E. coli cytoplasm in which a peptide of interest is co-expressed and enzymatically processed to epitope length for loading onto MHC; production of libraries of pMHC using plasmid libraries encoding multiple epitope peptides; production of pMHC multimers (i.e. tetramers, pentamers etc.) loaded with a peptide of interest, including libraries thereof, in the E. coli cytoplasm; profiling the peptide-specificity of an MHC allele of interest by generating libraries of pMHC multimers and subsequently eluting peptides from purified pMHC for MS/MS analysis; production of RNA-barcoded pMHC multimers, including libraries thereof, in the E. coli cytoplasm; production of virus-like-particles decorated with pMHC loaded with a peptide-of-interest and encapsulating an RNA barcode, including libraries thereof; utilizing the protein and nucleic acid compositions described herein for labeling antigen specific T cells via interaction with the T cell receptor; and single-cell RNA sequencing of single T cells bound with pMHC VLPs (or RNA-barcoded pMHC multimers) displaying cognate antigen in order to pair TCR sequences paired with cognate peptide identity.

In its various implementations, this method enables the scalable production of pMHC-VLP libraries that can be used as a direct substitute for pMHC tetramers (including DNA-barcoded tetramers) to characterize T cell specificity, and is directly compatible with existing single-cell RNA sequencing platforms, such as the 10× Genomics Chromium. The process consists of the following steps: 1) design a library of up to 10¹⁰ peptide sequences and order DNA oligonucleotides encoding these peptides (or use random nucleotides to produce degenerate sequences); 2) clone oligonucleotides into a plasmid that include the HLA allele of interest. In some implementations it may be required to sequence the library at this point to link peptide sequence to the RNA barcode; 3) express and purify a pooled library of RNA-barcoded pMHC multimers; 4) pan pMHC multimers against a library of T cells (i.e., up to 1 million T cells from human blood); 5) wash unbound pMHC multimers and load T cells for single-cell RNA sequencing. For each T cell, one can recover (1) the TCR sequence, (2) the transcriptome of the cell, and (3) the RNA barcode of any bound pMHC multimers. Moreover, this method allows unprecedented scale at extremely low cost, compared to current methods.

### Example 4

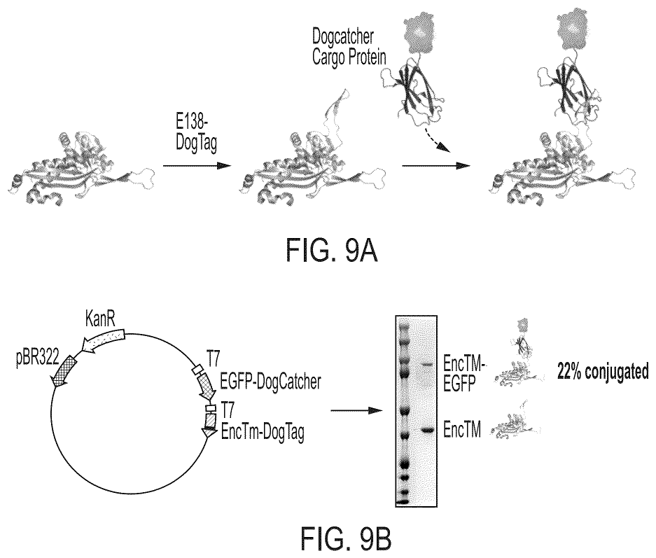

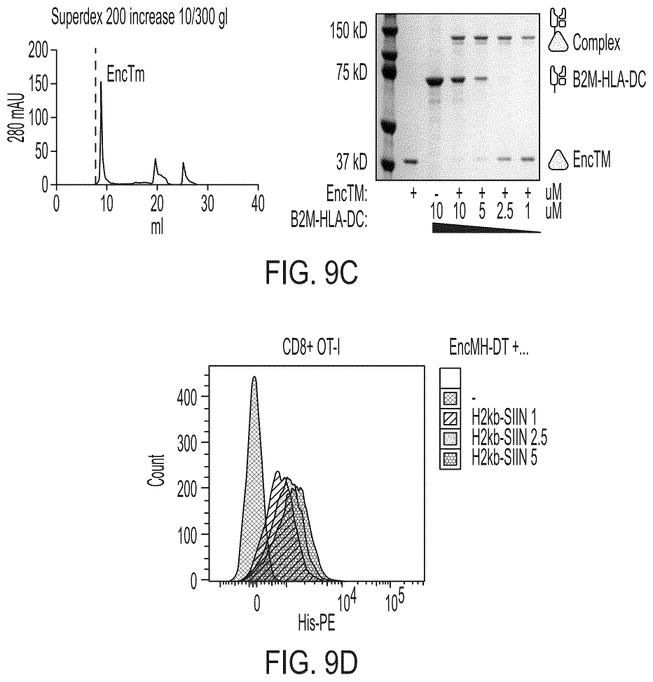

In some aspects of the disclosure, the virus-like particles or self-assembling nanocompartments disclosed herein are formed via non-viral self-assembling coat proteins. For example, a bacterial encapsulin protein derived from T. maritima may be used as a self-assembling coat protein (e.g., SEQ ID NO: 11). In some implementations, the encapsulin protein is engineered such that it is linked to a binding peptide, such as DogTag (e.g., SEQ ID NO: 12). To demonstrate the efficacy of a non-viral self-assembling coat protein in the generation VLPs, a DogTag peptide was inserted into a surface-accessible (e.g., hydrophilic) loop within the encapsulin protein after amino acid location E135 in SEQ ID NO: 11 to generate a DogTag-linked encapsulin protein (i.e., EncTM-DogTag), which was further modified to contain a His tag on the C-terminus of the protein. With this system, a cargo protein fused to the DogCatcher protein domain then forms an isopeptide bond between the DogTag and DogCatcher peptides, covalently linking the cargo with the EncTM-DogTag (see FIG. 9A). As proof-of-concept, EGFP was fused with the DogCatcher protein domain (EGFP-DogCatcher) and co-expressed on a vector with EncTM-DogTag, both of which were under the control of the constitutive T7 promoter. When expressed in E. coli cells, co-expression of EGFP-DogCatcher and EncTM-DogTag resulted in approximately 22% of the EncTM-DogTag proteins undergoing covalent conjugation with EGFP-DogCatcher via isopeptide bond to form EncTM-DogTag+EGFP-DogCatcher virus-like particles (FIG. 9B).

Further testing of the EncTM-DogTag system demonstrated its effectiveness with other physiologically relevant cargo proteins. An MHC protein comprising beta-2-microglobulin (B2M) and human leukocyte antigen (HLA) were fused with the DogCatcher protein domain (B2M-HLA-DC) and co-expressed with EncTM-DogTag containing a C-terminal His tag in E. coli cells. Size-exclusion chromatography demonstrated the proper assembly of the EncTM-DogTag monomers into larger species corresponding to assembled T=1 capsids encapsulating B2M-HLA-DC (see FIG. 9C, right; compare monomers of EncTM-DogTag (37 kDa) and B2M-HLA-DC (75 kDa) to the complex of EncTM-DogTag+B2M-HLA-DC (150 kD)).

These EncTM-DogTag+B2M-HLA-DC VLPs were then prepared with MHC class I H2Kb loaded with SIINFEKL peptides to test their ability to label specifically label T cells expressing the OT-I TCR (Kb/SIINFELK). Fluorescent labeling was performed a secondary anti-His antibody that recognized the His tag on the C-terminus of EncTM-DogTag, and readout via flow cytometry showed that SIINFEKL-reactive OT-I T cells were specifically labeled in a concentration-dependent manner (FIG. 9D), indicating the specificity of binding between the MHC-presented antigen and TCR.

### INCORPORATION BY REFERENCE

All patents and other publications, including documents, references, published patents, published patent applications, and co-pending patent applications cited throughout this application are, for example, described in the technology described herein. For purposes of describing and disclosing the methodology described in such publications that may be used in connection with the above, expressly incorporated herein by reference. These publications are provided solely for their disclosure prior to the filing date of the present application. Nothing in this regard should be construed as an admission that the inventors have no right to advance the date of such disclosure, based on the prior invention or for any other reason. All statements regarding the date or content of these documents are based on information available to the Applicant and do not give any approval as to the accuracy of the date or content of these documents.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for producing complexes between an antigen peptide and a major histocompatibility complex (MHC), comprising:
(a) providing an Escherichia coli cell under conditions suitable for expression, wherein the E. coli cell comprises one or more sequences encoding a MHC, one or more sequences encoding one or more heterologous oxidation enzymes, and a sequence encoding an antigen peptide; and
(b) isolating complexes between the antigen peptide and MHC (pMHC) from the E. coli cell.

2. The method of claim 1, wherein the one or more sequences encoding a MHC comprise:
a sequence encoding human leukocyte antigen (HLA) that is at least 70% identical to or is 100% identical to NCBI Reference Sequence: NM_002116.8; Gene ID: 3105; and/or
a sequence encoding beta-2-microglobulin (B2M) that is at least 70% identical to or is 100% identical to NCBI Reference Sequence: NM_004048.4; Gene ID: 567.

3. The method of claim 1 or claim 2, wherein the one or more sequences encoding one or more heterologous oxidation enzymes comprise a sequence encoding mitochondrial flavin adenine dinucleotide (FAD)-linked sulfhydryl oxidase (Erv1) and/or a sequence encoding protein disulfide isomerase (PDI).

4. The method of claim 3, wherein the sequence encoding Erv1 is derived from Saccharomyces cerevisiae; and
wherein the sequence encoding Erv1 is at least 70% identical to or is 100% identical to NCBI Reference Sequence: NM_001181158.3; Gene ID: 852916.

5. The method of claim 3 or claim 4, wherein the sequence encoding PDI is derived from Saccharomyces cerevisiae or Homo sapiens; and
wherein the sequence encoding PDI is at least 70% identical to or is 100% identical to NCBI Reference Sequences: NM_001178688.1 or NM_006849.4; Gene IDs: 852916 or 850314.

6. The method of any one of claims 1-5, wherein the sequence encoding an antigen peptide encodes an antigen peptide that is further modified by fusion to a protein label.

7. The method of claim 6, wherein the protein label is ubiquitin-like protein SMT3; and
wherein the sequence of SMT3 is at least 80% identical to or is 100% identical to NCBI Reference Sequence: NP_010798.1; Gene ID: 852122.

8. The method of claim 6 or claim 7, wherein the protein label is fused to the N-terminus of the antigen peptide.

9. The method of any one of claims 6-8, wherein the E. coli cell further comprises one or more sequences encoding a protease that removes the protein label.

10. The method of claim 9, wherein the one or more sequences encoding a protease encode for ubiquitin-like-specific protease 1 (Ulp1); and
wherein the one or more sequences encoding a protease is at least 70% identical to or is 100% identical to NCBI Reference Sequence: NM_001183834.1; Gene ID: 856087.

11. The method of any one of claims 1-10, wherein the antigen peptide is an antigen peptide randomly selected from a library of antigen peptides.

12. The method of any one of claims 1-11, wherein the one or more sequences encoding a MHC, the one or more sequences encoding one or more heterologous oxidation enzymes, and/or the sequence encoding an antigen peptide are comprised by one or more plasmids.

13. The method of any one of claims 9-12, wherein the one or more sequences encoding a protease are comprised by one or more plasmids.

14. The method of any one of claims 1-13, wherein the one or more sequences encoding a MHC, the one or more sequences encoding one or more heterologous oxidation enzymes, and/or the sequence encoding an antigen peptide are integrated into the E. coli genome.

15. The method of any one of claims 9-11 or claim 13, wherein the one or more sequences encoding a protease are integrated into the E. coli genome.

16. The method of any one of claims 1-15, wherein the one or more sequences encoding a MHC, the one or more sequences encoding one or more heterologous oxidation enzymes, and/or the sequence encoding an antigen peptide are operably linked to a constitutive promotor.

17. The method of any one of claims 9-11, claim 13, or claim 15, wherein the one or more sequences encoding a protease are operably linked to a constitutive promoter.

18. The method of any one of claims 1-15, wherein the one or more sequences encoding a MHC, the one or more sequences encoding one or more heterologous oxidation enzymes, and/or the sequence encoding an antigen peptide are operably linked to an inducible promotor.

19. The method of any one of claims 9-11, claim 13, or claim 15, wherein the one or more sequences encoding a protease are operably linked to an inducible promoter.

20. The method of any one of claims 1-19, wherein the method further comprises a step of assaying the isolated pMHC.

21. The method of claim 20, wherein assaying the isolated pMHC comprises purifying the isolated pMHC by affinity chromatography.

22. The method of claim 20 or claim 21, wherein assaying the isolated pMHC comprises separating the antigen peptide from the pMHC and/or analyzing the antigen peptide by mass spectrometry.

23. The method of any one of claims 1-22, wherein the isolated pMHC is multimeric.

24. A viral-like particle (VLP) conjugated to a pMHC, comprising:
(a) a VLP comprised of a self-assembling coat protein;
(b) a major histocompatibility complex (MHC); and
(c) an antigen peptide complexed with the MHC (pMHC),
wherein the self-assembling coat protein and MHC are modified by fusion to a binding protein and binding peptide, respectively, and
wherein the binding protein conjugates to the binding peptide.

25. The virus-like particle of claim 24, wherein the self-assembling coat protein is a viral self-assembling coat protein,
optionally a MS2 or PP7 bacteriophage coat protein, wherein the MS2 protein has at least 80% sequence identity to or is 100% identical to SEQ ID NO:1, and wherein the PP7 protein has at least 80% sequence identity to or is 100% identical to SEQ ID NO: 2.

26. The virus-like particle of any claim 24 or claim 25, wherein the binding peptide and the binding protein conjugate through a covalent bond.

27. The virus-like particle of claim 26, wherein the binding peptide is at least 80% identical to or is 100% identical to the sequence set forth in any one of SEQ ID NOs: 3-5 (e.g., SpyTag) and the binding protein is at least 80% identical to or is 100% identical to the sequence set forth in SEQ ID NO: 6 (e.g., SpyCatcher).

28. The virus-like particle of claim 26, wherein the binding peptide is at least 80% identical to or is 100% identical to the sequence set forth in SEQ ID NO: 9 (e.g., DogTag), and the binding protein is at least 80% identical to or is 100% identical to the sequence set forth in SEQ ID NO: 10 (e.g., DogCatcher).

29. The virus-like particle of any one of claims 24-28, wherein the VLP comprises up to 180 sites for conjugation with pMHC.

30. The virus-like particle of any one of claims 24-29, wherein the VLP is conjugated to up to 180 pMHCs.

31. The virus-like particle of any one of claims 24-30, wherein the antigen peptides of pMHCs complexed with the VLP are the same or different.

32. The virus-like particle of any one of claims 24-31, wherein the pMHCs are multimeric.

33. A method for producing viral-like particles (VLPs) conjugated to pMHC, comprising:
(a) providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide; and
(b) isolating VLPs conjugated to complexes between the antigen peptide and MHC (pMHC) from the E. coli cell.

34. The method of claim 33, wherein the one or more sequences encoding a MHC comprise:
a sequence encoding human leukocyte antigen (HLA) that is at least 70% identical to or is 100% identical to NCBI Reference Sequence: NM_002116.8; Gene ID: 3105; and/or
a sequence encoding beta-2-microglobulin (B2M) that is at least 70% identical to or is 100% identical to NCBI Reference Sequence: NM_004048.4; Gene ID: 567.

35. The method of claim 33 or claim 34, wherein the one or more sequences encoding one or more heterologous oxidation enzymes comprise a sequence encoding mitochondrial FAD-linked sulfhydryl oxidase (Erv1) and/or a sequence encoding protein disulfide isomerase (PDI).

36. The method of claim 35, wherein the sequence encoding Erv1 is derived from Saccharomyces cerevisiae; and
wherein the sequence encoding Erv1 is at least 70% identical to or is 100% identical to NCBI Reference Sequence: NM_001181158.3; Gene ID: 852916.

37. The method of claim 35 or claim 36, wherein the sequence encoding PDI is derived from Saccharomyces cerevisiae or Homo sapiens; and
wherein the sequence encoding PDI is at least 70% identical to or is 100% identical to NCBI Reference Sequences: NM_001178688.1 or NM_006849.4; Gene IDs: 852916 or 850314.

38. The method of any one of claims 33-37, wherein the sequence encoding an antigen peptide encodes an antigen peptide that is further modified by fusion to a protein label.

39. The method of claim 38, wherein the protein label is ubiquitin-like protein SMT3, and
wherein the sequence of SMT3 is at least 80% identical to or is 100% identical to NCBI Reference Sequence: NP_010798.1; Gene ID: 852122.

40. The method of claim 38 or claim 39, wherein the protein label is fused to the N-terminus of the antigen peptide.

41. The method of any one of claims 38-40, wherein the E. coli cell further comprises one or more sequences encoding a protease that removes the protein label.

42. The method of claim 41, wherein the one or more sequences encoding a protease encode for ubiquitin-like-specific protease 1 (Ulp1); and
wherein the one or more sequences encoding a protease is at least 70% identical to or is 100% identical to NCBI Reference Sequence: NM_001183834.1; Gene ID: 856087.

43. The method of any one of claims 33-42, wherein the antigen peptide is an antigen peptide randomly selected from a library of antigen peptides.

44. The method of any one of claims 33-43, wherein the self-assembling coat protein is a viral self-assembling coat protein,
optionally a MS2 or PP7 bacteriophage coat protein, wherein the MS2 protein has at least 80% sequence identity to or is 100% identical to SEQ ID NO:1, and wherein the PP7 protein has at least 80% sequence identity to or is 100% identical to SEQ ID NO: 2.

45. The method of any one of claims 33-44, wherein the VLPs each comprise up to 180 sites for conjugation with pMHC.

46. The method of any one of claims 33-45, wherein the pMHCs are multimeric.

47. The method of any of claims 33-46, wherein the binding peptide and the binding protein conjugate through a covalent bond.

48. The method of claim 47, wherein the binding peptide is at least 80% identical to or is 100% identical to the sequence set forth in any one of SEQ ID NOs: 3-5 (e.g., SpyTag) and the binding protein is at least 80% identical to or is 100% identical to the sequence set forth in SEQ ID NO: 6 (e.g., SpyCatcher).

49. The method of claim 47, wherein the binding peptide is at least 80% identical to or is 100% identical to the sequence set forth in SEQ ID NO: 9 (e.g., DogTag), and the binding protein is at least 80% identical to or is 100% identical to the sequence set forth in SEQ ID NO: 10 (e.g., DogCatcher).

50. The method of any one of claims 33-49, wherein the binding peptide is fused to the C-terminus of the MHC and/or the binding protein is fused to the C-terminus of the self-assembling coat protein.

51. The method of any one of claims 33-50, wherein the one or more sequences encoding a MHC, the one or more sequences encoding one or more heterologous oxidation enzymes, the sequence encoding an antigen peptide, and/or the sequence encoding the self-assembling coat protein are comprised by one or more plasmids.

52. The method of any one of claims 33-51, wherein the one or more sequences encoding a MHC, the one or more sequences encoding one or more heterologous oxidation enzymes, the sequence encoding an antigen peptide, and/or the sequence encoding the self-assembling coat protein are integrated into the E. coli genome.

53. The method of any one of claims 33-52, wherein the one or more sequences encoding a MHC, the one or more sequences encoding one or more heterologous oxidation enzymes, the sequence encoding an antigen peptide, and/or the sequence encoding the self-assembling coat protein are operably linked to a constitutive promotor.

54. The method of any one of claims 33-52, wherein the one or more sequences encoding a MHC, the one or more sequences encoding one or more heterologous oxidation enzymes, the sequence encoding an antigen peptide, and/or the sequence encoding the self-assembling coat protein are operably linked to an inducible promotor.

55. A method for identifying pMHC-TCR pairs, comprising:
(a) providing an E. coli cell under conditions suitable for expression, wherein the E. coli cell comprises one or more sequences encoding a major histocompatibility complex (MHC) modified by fusion with a binding peptide, one or more sequences encoding one or more heterologous oxidation enzymes, a sequence encoding an antigen peptide, and a sequence encoding a self-assembling coat protein modified by fusion with a binding protein capable of conjugating to the binding peptide;
(b) isolating virus-like particles (VLPs) conjugated to complexes between the antigen peptide and MHC (pMHC) from the E. coli cell;
(c) contacting the isolated VLPs with a population of T cells;
(d) sequencing the population of T cells; and
(e) determining pMHC-T cell receptor (TCR) cognate pairs by identifying TCR-encoding sequences and antigen peptide-encoding sequences comprised by each T cell within the population of T cells.

56. The method of claim 55, wherein the one or more sequences encoding a MHC comprise:
a sequence encoding human leukocyte antigen (HLA) that is at least 70% identical to or is 100% identical to NCBI Reference Sequence: NM_002116.8; Gene ID: 3105; and/or
a sequence encoding beta-2-microglobulin (B2M) that is at least 70% identical to or is 100% identical to NCBI Reference Sequence: NM_004048.4; Gene ID: 567.

57. The method of claim 55 or claim 56, wherein the one or more sequences encoding one or more heterologous oxidation enzymes comprise a sequence encoding mitochondrial flavin adenine dinucleotide (FAD)-linked sulfhydryl oxidase (Erv1) and/or a sequence encoding protein disulfide isomerase (PDI).

58. The method of claim 57, wherein the sequence encoding Erv1 is derived from Saccharomyces cerevisiae, and
wherein the sequence encoding Erv1 is at least 70% identical to or is 100% identical to NCBI Reference Sequence: NM_001181158.3; Gene ID: 852916.

59. The method of claim 57 or 58, wherein the sequence encoding PDI is derived from Saccharomyces cerevisiae or Homo sapiens; and
wherein the sequence encoding PDI is at least 70% identical to or is 100% identical to NCBI Reference Sequences: NM_001178688.1 or NM_006849.4; Gene IDs: 852916 or 850314.

60. The method of any one of claims 55-59, wherein the sequence encoding an antigen peptide encodes an antigen peptide that is further modified by fusion to a protein label.

61. The method of claim 60, wherein the protein label is ubiquitin-like protein SMT3 and
wherein the sequence of SMT3 is at least 80% identical to or is 100% identical to NCBI Reference Sequence: NP_010798.1; Gene ID: 852122.

62. The method of claim 60 or claim 61, wherein the protein label is fused to the N-terminus of the antigen peptide.

63. The method of any one of claims 60-62, wherein the E. coli cell further comprises one or more sequences encoding a protease that removes the protein label.

64. The method of claim 63, wherein the one or more sequences encoding a protease encode for ubiquitin-like-specific protease 1 (Ulp1); and
wherein the one or more sequences encoding a protease is at least 70% identical to or is 100% identical to NCBI Reference Sequence: NM_001183834.1; Gene ID: 856087.

65. The method of any one of claims 55-64, wherein the antigen peptide is an antigen peptide randomly selected from a library of antigen peptides.

66. The method of any one of claims 55-65, wherein the self-assembling coat protein is a viral self-assembling coat protein,
optionally a MS2 or PP7 bacteriophage coat protein, wherein the MS2 protein has at least 80% sequence identity to or is 100% identical to SEQ ID NO:1, and wherein the PP7 protein has at least 80% sequence identity to or is 100% identical to SEQ ID NO: 2.

67. The method of any one of claims 55-66, wherein the VLPs each comprise at least 90 sites for conjugation with pMHC.

68. The method of any one of claims 55-67, wherein the pMHCs are multimeric.

69. The method of any of claims 55-68, wherein the binding peptide and the binding protein conjugate through a covalent bond.

70. The method of claim 68, wherein the binding peptide is at least 80% identical to or is 100% identical to the sequence set forth in any one of SEQ ID NOs: 3-5 (e.g., SpyTag) and the binding protein is at least 80% identical to or is 100% identical to the sequence set forth in SEQ ID NO: 6 (e.g., SpyCatcher).

71. The method of claim 68, wherein the binding peptide is at least 80% identical to or is 100% identical to the sequence set forth in SEQ ID NO: 9 (e.g., DogTag), and the binding protein is at least 80% identical to or is 100% identical to the sequence set forth in SEQ ID NO: 10 (e.g., DogCatcher).

72. The method of any one of claims 55-71, wherein the binding peptide is fused to the C-terminus of the MHC and/or the binding protein is fused to the C-terminus of the self-assembling coat protein.

73. The method of any one of claims 55-72, wherein the one or more sequences encoding a MHC, the one or more sequences encoding one or more heterologous oxidation enzymes, the sequence encoding an antigen peptide, and/or the sequence encoding the self-assembling coat protein are comprised by one or more plasmids.

74. The method of any one of claims 55-73, wherein the one or more sequences encoding a MHC, the one or more sequences encoding one or more heterologous oxidation enzymes, the sequence encoding an antigen peptide, and/or the sequence encoding the self-assembling coat protein are integrated into the E. coli genome.

75. The method of any one of claims 55-74, wherein the one or more sequences encoding a MHC, the one or more sequences encoding one or more heterologous oxidation enzymes, the sequence encoding an antigen peptide, and/or the sequence encoding the self-assembling coat protein are operably linked to a constitutive promotor.

76. The method of any one of claims 55-74, wherein the one or more sequences encoding a MHC, the one or more sequences encoding one or more heterologous oxidation enzymes, the sequence encoding an antigen peptide, and/or the sequence encoding the self-assembling coat protein are operably linked to an inducible promotor.

77. The method of any one of claims 55-76, wherein the sequence encoding the antigen peptide further encodes a barcode sequence.

78. The method of claim 77, wherein the barcode sequence is a DNA or RNA sequence.

79. The method of claim 77 or 78, wherein the barcode sequence is encapsulated by the VLPs.

80. The method of claim 79, wherein the step of determining pMHC-T cell receptor (TCR) cognate pairs comprises identifying TCR-encoding sequences and barcode sequences comprised by each T cell within the population of T cells.

81. The method of any one of claims 55-80, wherein the step of sequencing the population of T cells comprises single cell RNA sequencing (RNA-seq).

82. The method of any one of claims 55-81, wherein the antigen peptide is an antigen peptide from a cancer cell.

83. The method of any one of claims 55-81, wherein the antigen peptide is an antigen peptide from a bacterial cell, parasite cell, or virus.

84. The method of any one of claims 55-83, wherein the antigen peptide is an antigen peptide of no known pMHC-TCR cognate pair.

85. The virus-like particle of claim 24, wherein the self-assembling coat protein is a non-viral self-assembling coat protein.

86. The virus-like particle of claim 85, wherein the non-viral self-assembling coat protein is a bacterial encapsulin protein derived from Thermotoga maritima.

87. The virus-like particle of claim 86, wherein the encapsulin protein is extended at the N-terminus, optionally with an RNA-binding domain.

88. The virus-like particle of claim 87, wherein the RNA-binding domain comprises a RNA-binding peptide that binds RNA in a sequence-independent manner.

89. The virus-like particle of claim 87, wherein the RNA-binding domain comprises a RNA-binding peptide that binds RNA in a sequence-dependent manner.

90. The virus-like particle of claim 88 or 89, wherein the RNA-binding peptide is derived from a P22 bacteriophage N protein.

91. The virus-like particle of claim 86, wherein the encapsulin protein comprises an amino acid sequence that is at least 70% identical to or is 100% identical to the sequence set forth in SEQ ID NOs: 11 or 12.

92. The method of claim 33, wherein the self-assembling coat protein is a non-viral self-assembling coat protein.

93. The method of claim 92, wherein the non-viral self-assembling coat protein is a bacterial encapsulin protein derived from Thermotoga maritima.

94. The method of claim 93, wherein the encapsulin protein is extended at the N-terminus, optionally with an RNA-binding domain.

95. The method of claim 94, wherein the RNA-binding domain comprises a RNA-binding peptide that binds RNA in a sequence-independent manner.

96. The method of claim 94, wherein the RNA-binding domain comprises a RNA-binding peptide that binds RNA in a sequence-dependent manner.

97. The method of claim 95 or 96, wherein the RNA-binding peptide is derived from a P22 bacteriophage N protein.

98. The method of claim 93, wherein the encapsulin protein comprises an amino acid sequence that is at least 70% identical to or is 100% identical to the sequence set forth in SEQ ID NOs: 11 or 12.

99. The method of claim 55, wherein the self-assembling coat protein is a non-viral self-assembling coat protein.

100. The method of claim 99, wherein the non-viral self-assembling coat protein is a bacterial encapsulin protein derived from Thermotoga maritima.

101. The method of claim 100, wherein the encapsulin protein is extended at the N-terminus, optionally with an RNA-binding domain.

102. The method of claim 101, wherein the RNA-binding domain comprises a RNA-binding peptide that binds RNA in a sequence-independent manner.

103. The method of claim 101, wherein the RNA-binding domain comprises a RNA-binding peptide that binds RNA in a sequence-dependent manner.

104. The method of claim 102 or claim 103, wherein the RNA-binding peptide is derived from a P22 bacteriophage N protein.

105. The method of claim 100, wherein the encapsulin protein comprises an amino acid sequence that is at least 70% identical to or is 100% identical to the sequence set forth in SEQ ID NOs: 11 or 12.


SEQUENCE LISTING (15 sequences)

SEQ ID NO: 1 - protein, 130 aa, unidentified
  source 1..130: note = Escherichia phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 2 - protein, 128 aa, unidentified
  source 1..128: note = Pseudomonas phage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 3 - protein, 13 aa, Synthetic construct
AHIVMVDAYKPTK

SEQ ID NO: 4 - protein, 14 aa, Synthetic construct
VPTIVMVDAYKRYK

SEQ ID NO: 5 - protein, 16 aa, Synthetic construct
RGVPHIVMVDAYKRYK

SEQ ID NO: 6 - protein, 113 aa, Synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 7 - protein, 113 aa, Synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 8 - protein, 113 aa, Synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 9 - protein, 23 aa, Synthetic construct
DIPATYEFTDGKHYITNEPIPPK

SEQ ID NO: 10 - protein, 105 aa, Synthetic construct
MKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 11 - protein, 265 aa, unidentified [sequence omitted]

SEQ ID NO: 12 - protein, 306 aa, Synthetic construct
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEGGGGSDIPATYEFTDGKHYITNEPIPPKGGGGSERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKFSGHHHHHH

SEQ ID NO: 13 - protein, 5 aa, synthetic construct
GGGGS

SEQ ID NO: 14 - protein, 8 aa, synthetic construct
SIINFEKL

SEQ ID NO: 15 - protein, 6 aa, synthetic construct
HHHHHH